(19) United States
(12) Patent Application Publication
(10) Pub. No.: US 2009/0229044 A1
(43) Pub. Date: Sep. 17, 2009

(54) Apparatus for removing debris from a lens

(76) Inventor: Jason Gregory Gill, Whitford (NZ)

Correspondence Address: ABELMAN, FRAYNE & SCHWAB, 666 THIRD AVENUE, 10TH FLOOR, NEW YORK, NY, 10017, US

(21) Appl. No.: 12/381,096

(22) Filed: Mar. 6, 2009

(30) Foreign Application Priority Data
Mar. 7, 2008 (NZ) 566513

(51) Int. Cl. A61F 9/02

(52) U.S. Cl. 2/434; 2/438

(57) ABSTRACT

A method and apparatus for removing debris from a transparent film across a permanent lens. The apparatus includes a transparent film perforated at predetermined intervals along the length of the film, a canister for holding the transparent film, the canister being adapted for placement adjacent one side of the lens, and a film engagement means for engaging a free end of the transparent film. The engagement means is adapted for placement adjacent an opposite side of the permanent lens, thereby allowing the transparent film to extend substantially across the permanent lens, wherein the transparent film is partially supported in position by lying flush against the permanent lens.

### TECHNICAL FIELD

The present invention relates generally to protective eyewear and, more particularly, to using transparencies to remove debris from a lens of the eyewear.

### BACKGROUND

The use of eyewear to protect a wearer's eyes is well known. The primary protection is provided by a lens of the eyewear. However, debris typically accumulates on the lens, thereby obscuring the vision of the wearer.

In many instances it is not possible for the wearer to remove the eyewear to clean the lens as such would be hazardous to the wearer. Also, situations exist where the wearer is not able to spend time wiping the debris of the lens. One such a situation is where goggles are used by a motocross rider to prevent mud and dust from entering its eyes. Especially in a race situation where the rider is not able to stop to clean the lens, there is a need for a mechanism to clean the lens from debris which does not require the goggles to be removed, and allows the rider to clean the lens with a minimum of interruption from the race situation.

Transparent covers for the lens of the eyewear have been proposed. Such transparent covers are placed over the lens of the eyewear, and can be removed or changed periodically when the covers become obstructed. By removing or changing the transparent cover, the debris is also removed. For the transparent covers to be useful, especially in race situations, it is a requirement that the transparent cover should be able to be changed or removed with a minimum interruption.

A system exists which provides a plurality of transparent covers superposed over one another, and over the lens of goggles. Each transparent cover is provided with a tab. When the outermost transparent cover becomes obscured, the wearer is able to remove that outermost transparent cover by grabbing and pulling its tab way from the lens. While this system has generally been successful, this system has a number of drawbacks. One drawback of this system is that it is often difficult for the wearer to grab the tab of the outermost transparent cover, especially during excessive movement associated with some activities. As a result, the wearer often removes more than one transparent cover simultaneously. Another drawback is that a large number of superposed covers affect the transparency of the eyewear. The wearer thus has to either limit the number of transparent covers, or sacrifice visibility. Yet another drawback is that dust and/or droplets accumulate between the transparent covers, which further affect the vision of the wearer.

Another system exists which includes rollers on respective sides of the lens. A transparent film extends from one roller to the other. When the segment of the transparent film overlying the lens becomes dirty, the wearer advances the transparent film by at least the length of the lens. A drawback with this system is that the receiving roller includes a scrapper to scrape debris from the transparent film to prevent the same from entering the receiving roller. By allowing debris to enter the receiving roller the ability to receive the film would be severely limited. As a result of the scraper, debris accumulates on the lens on the side of the receiving roller. Also, the mechanism employed to advance the film is often complex, resulting in undesirable failures.

### SUMMARY OF INVENTION

The present invention is directed to overcoming one or more of the above problems. According to one aspect of the present invention there is provided an apparatus for removing debris from a lens, said apparatus comprising a transparent film perforated at predetermined intervals along the length of said film; a canister for holding said transparent film, said canister being adapted for placement adjacent one side of the lens; and a film engagement means for engaging a free end of said transparent film, said film engagement means being adapted for placement adjacent an opposite side of the lens, thereby allowing the transparent film to extend substantially across the lens.

Preferably a free end of said transparent film is provided with one ore more holes associated with each perforation, and said film engagement means is one or more post members for engaging said transparent film at said one or more holes.

Preferably said film engagement means further includes a guide for guiding said film onto said one or more post members.

Preferably said film, when perforated, forms an elongate tab.

Preferably said apparatus further comprises at least one channel extending at least in part from a canister side to a film engagement means side of the lens for restricting movement of said film in at least one direction.

According to another aspect of the present invention there is provided a method of removing debris from a lens, said method comprising the steps of providing a transparent film perforated at predetermined intervals along the length of said film; providing a canister for holding said transparent film, said canister being adapted for placement adjacent one side of the lens; providing a film engagement means for engaging a free end of said transparent film, said film engagement means being adapted for placement adjacent an opposite side of the lens, thereby allowing the transparent film to extend substantially across the lens; releasing said free end from said film engagement means; advancing said transparent film across said lens; reengaging said transparent film with said film engagement means; and pulling on said free end allowing said transparent film to tear at a perforation on a distal side of said film engagement means.

Other aspects are also disclosed.

### BRIEF DESCRIPTION OF DRAWINGS

Further aspects of the present invention will become apparent from the ensuing description which is given by way of example only and with reference to the accompanying drawings in which:

FIG. 1 shows an embodiment of the present invention in the form of motorcycle goggles;

FIG. 2 shows an exploded view of the goggles shown in FIG. 1;

FIG. 3 shows a more detailed view of one side of the goggles of FIG. 1;

FIG. 4 shows a front view of the goggles shown in FIG. 1; and

FIGS. 5 and 6 show cross section views of the sides of a frame of the goggles of FIG. 1.

### DETAILED DESCRIPTION

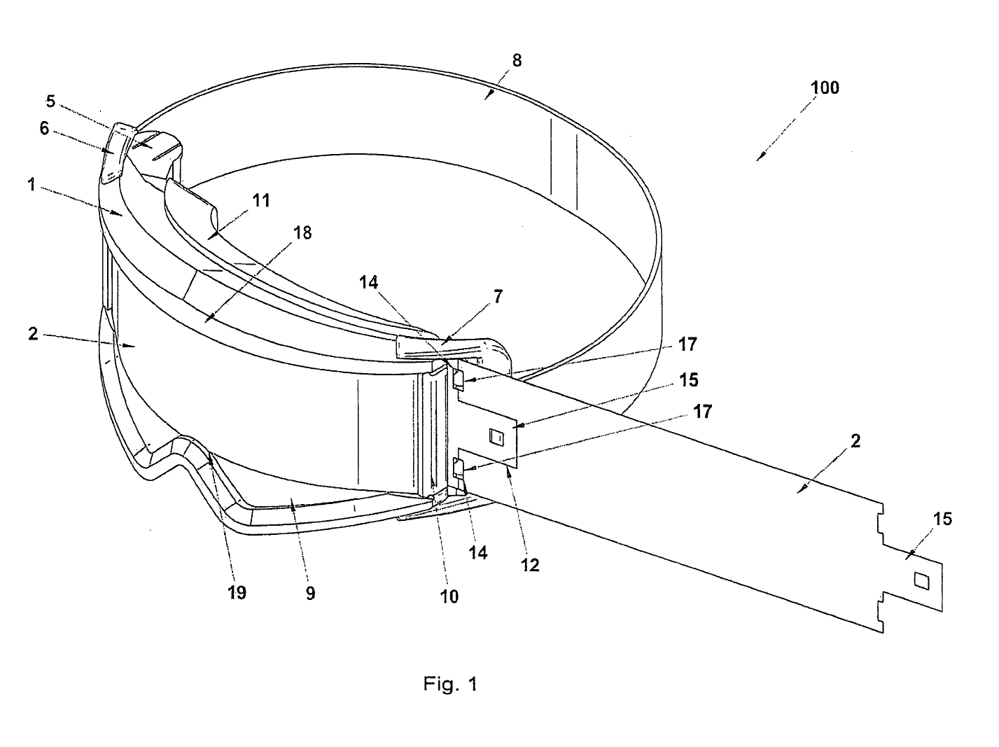

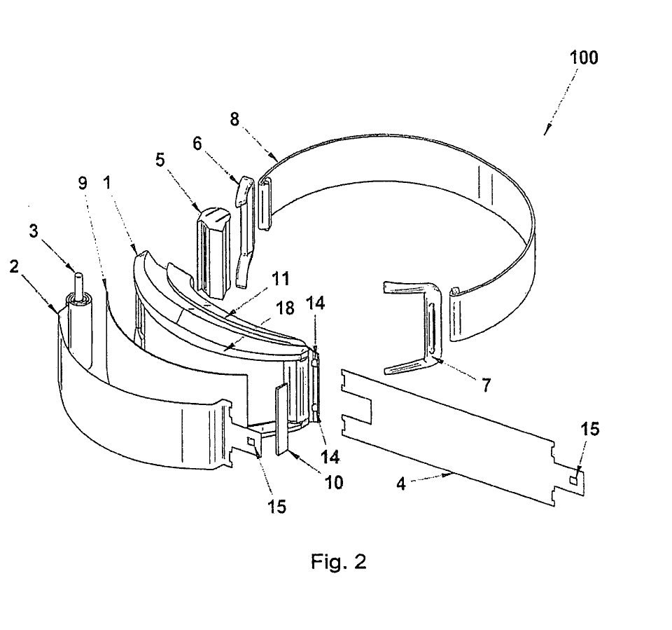

FIG. 1 shows a perspective view of an embodiment of the present invention in the form of motorcycle goggles 100. More particularly, the goggles 100 are for use during “off-road” and/or motocross events. FIG. 2 shows an exploded view of the goggles 100 shown in FIG. 1.

Referring to FIGS. 1 and 2, the goggles 100 have a frame 1 holding a transparent semi-rigid lens 9. An elastic head strap 8 is attached to the frame 1 by means of clips 6 and 7, allowing the goggles 100 to be secured to the head of a wearer. A layer of foam 11 is provided on the inside of the frame 1 for providing comfort to the wearer.

Adjacent one end of the lens 9 there is mounted a canister 5 secured to the frame 1. The canister 5 houses a roll of transparent film 2 wound around a spool 3 (not visible in FIG. 1). The canister 5 is formed by a tubular housing having a slot close to and parallel to the lens 9. The slot defines an exit for the transparent film 2, and serves to guide the transparent film 2 onto one end of the lens 9. The roll tends to expand within the canister 5, thereby frictionally engaging the interior of the canister 5. This frictional engagement provides sufficient resistance on the transparent film 2 to prevent the film 2 from exiting the canister 5 in the absence of a pulling force.

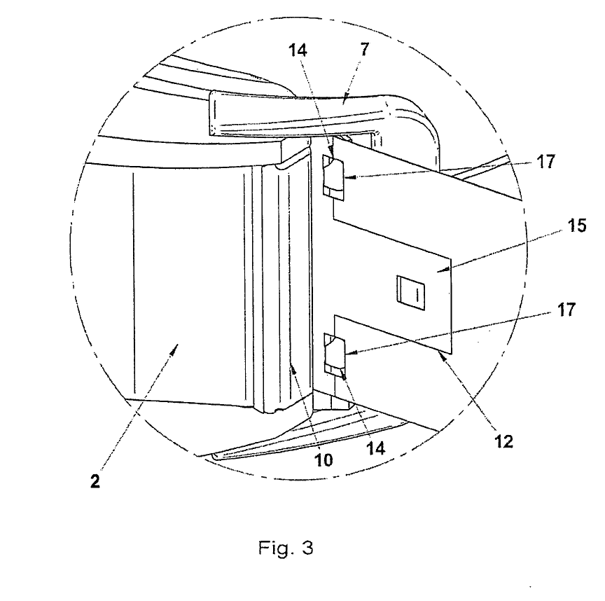

The film 2 extends from the canister 5 across the lens 9 to post members 14 located on an opposite side of the lens 9 from the canister 5. FIG. 3 shows a more detailed view of the side of the goggles 100 containing the post members 14. The post members 14 are secured to the frame 1. Also provided in association with the post members 14 is guide 10 for receiving the transparent film 2 and for guiding the same towards the post members 14. The guide 10 creates a sharp bend in the film 2 as the guide 10 guides the film 2 towards the post member 14. This bending action creates additional friction forces on the film 2, thereby preventing the film 2 from moving in the absence of a pulling force.

As shown in FIGS. 1 and 3, the transparent film 2 is provided with perforations 12 at regular predetermined intervals. The intervals are greater or equal to the width of the lens 9. The perforations 12 form a tab 15 at the free end of the film 2 opposite the canister 5. Holes 17 are provided on the film 2 and in association with the perforations 12. The film 2, and in particular the holes 17 of the film 2, is engaged by the post members 14 when the film 2 is pulled through the guide 10 and over the post members 14.

Referring again to FIGS. 1 and 2, a C-shaped channel 18 is formed on the upper side of the frame 1. The upper side of the film 2, when extended across the lens 9, fits inside the channel 18. A gap 19 (not visible in FIG. 2) is formed on the lower side of the frame for receiving a lower side of the film 2 when extended across the lens 9. The channel 18 and gap 19 serve to guide the film 2 across the lens 9, and also to restrain vertical movement of the film 2 relative to the lens 9.

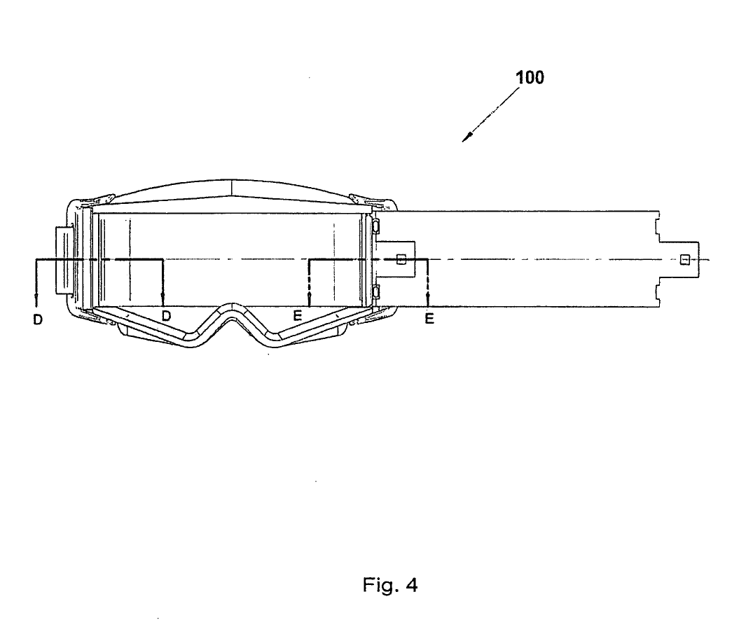

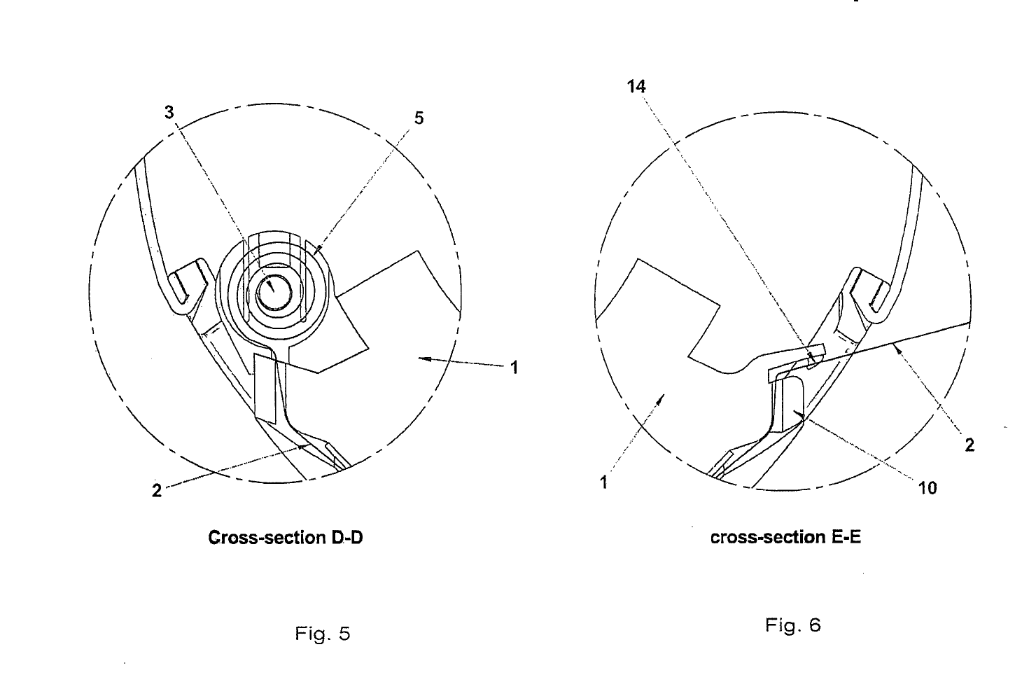

FIG. 4 shows a front view of the goggles shown in FIG. 1. FIGS. 5 and 6 show cross section views of the sides of the frame 1 to more clearly illustrate the path of the film 2 from the spool 3 of the canister 5 on the one side, to the guide 10 and post members 14 on the other.

When the portion of the film 2 overlying the lens 9 is obscured by debris, the wearer of the goggles 100 grabs the tab 15 and pulls a length of film 2 across the lens 9. The pulling force unwinds the length of film 170 from the spool 3 of the canister 5. More particularly, the film 170 is advanced until the next holes 17 align with the post members 14, at which point the post members 14 would engage the film 2. By continuing the lateral pulling force the film 2 tears at the perforation 12 associated with the next holes 17. A complete change of the portion of the film 2 overlaying the lens 9 results, leaving a lens clear of any debris.

Aspects of the present invention have been described by way of example only and it should be appreciated that modifications and additions may be made thereto without departing from the scope thereof as defined in the appended claims.

For example, even though the proffered embodiment is in the form of motorcycle goggles 100, the present invention may also be applied to other lenses, such as those on helmets.

When comparing embodiments of the present invention with the plurality of superposed transparent covers discussed in the “Background” section, the present invention provides only a single layer of film over the lens, thereby improving visibility. The drawback that exists with the prior art system of removing more than one transparent cover simultaneously has also been addressed.

When comparing embodiments of the present invention with the prior art system having rollers on respective sides discussed in the “Background” section, the present invention avoids the complex mechanism for advancing the film, thereby avoiding undesirable failures. Also, in the embodiment of the present invention described herein debris does not accumulate on one side of the lens, thereby providing a wider clear view.

While the foregoing is directed to embodiments of the present invention, other and further embodiments of the invention may be devised without departing from the basic scope thereof, and the scope thereof is determined by the claims that follow.

1. Apparatus for removing debris from moving a transparent film across a permanent lens, said apparatus comprising:
a transparent film perforated at predetermined intervals along the length of said film;
a canister for holding said transparent film, said canister being adapted for placement adjacent one side of the lens; and
a film engagement means for engaging a free end of said transparent film, said film engagement means being adapted for placement adjacent an opposite side of the permanent lens, thereby allowing the transparent film to extend substantially across the permanent lens; wherein the transparent film is partially supported in position by lying flush against the permanent lens.

2. Apparatus according to in claim 1 wherein a free end of said transparent film is provided with one or more holes associated with each perforation; and said film engagement means is one or more post members for engaging said transparent film at said one or more holes.

3. Apparatus according to in claim 2 wherein said film engagement means further includes a guide for guiding said film onto said one or more post members.

4. Apparatus according to claim 1 wherein said film, when perforated, forms an elongate tab.

5. Apparatus according to claim 1 further comprising at least one channel extending at least in part from a canister side to a film engagement means side of the lens for restricting movement of said film in at least one direction.

6. A method of removing debris from moving a transparent film across a permanent lens, said method comprising the steps of:
providing a transparent film perforated at predetermined intervals along the length of said film;
providing a canister for holding said transparent film, said canister being adapted for placement adjacent one side of the permanent lens;
providing a film engagement means for engaging a free end of said transparent film, said film engagement means being adapted for placement adjacent an opposite side of the permanent lens, thereby allowing the transparent film to extend substantially across the permanent lens; wherein the transparent film is partially supported in position by lying flush against the permanent lens;
releasing said free end from said film engagement means;
advancing said transparent film across said permanent lens;
re-engaging said transparent film with said film engagement means; and
pulling on said free end allowing said transparent film to tear at a perforation on a distal side of said film engagement means.

7. Apparatus according to claim 2 wherein said film, when perforated, forms an elongate tab.

8. Apparatus according to claim 3 wherein said film, when perforated, forms an elongate tab.

9. Apparatus according to claim 2 further comprising at least one channel extending at least in part from a canister side to a film engagement means side of the lens for restricting movement of said film in at least one direction.

10. Apparatus according to claim 3 further comprising at least one channel extending at least in part from a canister side to a film engagement means side of the lens for restricting movement of said film in at least one direction.

11. Apparatus according to claim 4 further comprising at least one channel extending at least in part from a canister side to a film engagement means side of the lens for restricting movement of said film in at least one direction.
